(19) United States
(12) Patent Application Publication
(10) Pub. No.: US 2014/0357584 A1
(43) Pub. Date: Dec. 4, 2014

(54) ARYL NAPHTHALIDE LIGNANS AS ANTI-HIV AGENTS

(71) Applicant: THE BOARD OF TRUSTEES OF THE UNIVERSITY OF ILLINOIS, Urbana, IL (US)

(72) Inventors: Hongjie Zhang, Hong Kong (CN); Djaja Soejarto, Lombard, IL (US); Lijun Rong, Westmont, IL (US); Harry H.S. Fong, Glen Ellyn, IL (US); Emily Rumschlag-Booms, Chicago, IL (US)

(73) Assignee: THE BOARD OF TRUSTEES OF THE UNIVERSITY OF ILLINOIS, Urbana, IL (US)

(21) Appl. No.: 14/235,870

(22) PCT Filed: Jul. 27, 2012
(86) PCT No.: PCT/US2012/048657

§ 371 Date: Aug. 19, 2014

Related U.S. Application Data
(60) Provisional application No. 61/513,119, filed on Jul. 29, 2011.

(51) Int. Cl. C07H 17/04

(52) U.S. Cl. CPC C07H 17/04
USPC 514/27

(57) ABSTRACT

Provided herein are glycosidic aryl naphthalide lignans compounds, such as justiprocumin A isolated from the plant Justicia gendarussa Burm f. (Acanthaceae), which are effective in the treatment of AIDS and HIV infections.

### CROSS-REFERENCE TO RELATED APPLICATIONS

This application claims priority from U.S. Provisional Patent Application Ser. No. 61/513,119 filed Jul. 29, 2011, which is hereby incorporated by reference in its entirety.

### STATEMENT REGARDING FEDERALLY SPONSORED RESEARCH

This invention was made with government support under Grant 1 UO1-TW01015-01 awarded by the National Institutes of Health administered by the Fogarty International Center. The United States Government has certain rights in the invention.

### BACKGROUND OF THE INVENTION

The present invention relates to anti-HIV agents originally derived from natural sources. More particularly, the agents are derivatives of aryl naphthalide lignans. It is a goal of the present invention to provide aryl naphthalide lignan compounds having anti-HIV activity.

AIDS has become a worldwide epidemic since its first report in 1981 in the US. In a UNAIDS (Uniting the world against AIDS) report, approximately 25 million people have died from AIDS (acquired immunodeficiency syndrome) and an estimated 33 million individuals are infected with the human immunodeficiency virus (HIV). AIDS is presently the leading cause of death in Africa and ranked as the fourth leading cause of death worldwide behind heart disease, stroke and respiratory infections. In Africa, it is estimated that more than 22 million people live with HIV and more than 2 million people are killed each year. According to the report by Mathers and Loncar (2006), AIDS is projected to be the third leading cause of death in 2030, and it is estimated that a total of nearly 120 million people could die from AIDS in the next 25 years (Mathers C D, Loncar D. PLoS Med 2006; 3: 2011-2030).

There are many anti-HIV therapeutic drugs currently in clinical use. The first anti-HIV drug, AZT (zidovudine), was developed by GlaxoSmithKline and approved in 1987. More anti-HIV drugs were introduced in the 1990s and 2000s. Today, more than 20 anti-HIV drugs are available on the market for HIV infected patients. Among these drugs, AZT, ddI (didanosine), ddC (zalcitabine), d4T (stavudine), 3TC (lamivudine), abacavir, tenofovir and emtricitabine were developed as nucleoside/nucleotide reverse transcriptase (RT) inhibitors; nevirapine, delavirdine, efavirenz and etravirine as nonnucleoside reverse transcriptase inhibitors; saquinavir, indinavir, ritonavir, nelfinavir, amprenavir, lopinavir/ritonavir, fosamprenavir, atazanavir, tipranavir and darunavir as protease inhibitors; enfuvirtide and maraviroc as entry and fusion inhibitors; and raltegravir as an integrase inhibitor. Although these drugs have significantly extended the life span of HIV-positive people in wealthy countries (Thayer A M. C&EN 2008; September 22: 29-36.), they are non-curative, and have the problems of side effects and diminishing effectiveness due to the development of viral resistance (Cos P, Maes L, Berghe D V, Hermans N, Pieters L, Vlietinck A. Journal of Natural Products 2004; 67: 284-293.). The fact that there currently are no drugs capable of curing, nor vaccine available to prevent this viral diseases, the discovery and development of new anti-HIV drugs are very much needed.

Aryl naphthalide lignans are compounds which comprise a structure of 2,3-dimethyl-1-phenyl-naphthalene. The compounds occur naturally and can also be synthesized. The structure of aryl naphthalide lignan itself is shown below:

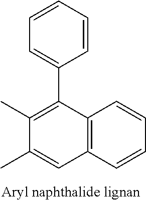 [SMILES: Cc1cc2ccccc2c(-c2ccccc2)c1C]

Some aryl naphthalide lignans have been reported to have anti-HIV activity in the literature (1. Chang C W, Lin M T, Lee S S, Liu KCSC, Hsu F L, Lin J Y. Antiviral Research 1995; 27: 367-74. 2. Sagar K S, Chang C C, Wang W K, Lin J Y, Lee S S. Bioorganic & Medicinal Chemistry 2004, 12:4045-4054. 3. Tuchinda P, Komsakulkam J, Pohmakotr M, Kongsaeree P, Prabpai S, Yoosook C, Kasisit J, Napaswad C, Sophasan S, Reutrakul V. Journal of Natural Products 2008; 71: 655-663.). However, none of the reported anti-HIV active aryl naphthalide lignans contain sugar units.

### SUMMARY OF THE INVENTION

The present invention is based, at least in part, on the discovery that glycosidic aryl naphthalide lignans compounds, such as justiprocumin A isolated from the plant Justicia gendarussa Burm.f. (Acanthaceae), that are effective in the treatment of AIDS and HIV infections.

Accordingly, a first aspect of the invention is an aryl naphthalide lignan compound or a pharmaceutically acceptable salt or pro-drug thereof, for use in the treatment, prevention or delay of progression of a HIV infection in a patient. The aryl naphthalide lignan compound may be a glycosidic aryl naphthalide lignan compound.

A second aspect of the invention is a pharmaceutical formulation comprising an aryl naphthalide lignan compound, for example a glycosidic aryl naphthalide lignan compound, or a pharmaceutically acceptable salt or prodrug thereof, for use in the treatment, prevention or delay of progression of a HIV infection in a patient.

Another aspect of the invention concerns the use of an extract or a fraction made from plant material containing one or more aryl naphthalide lignan compounds for use in the treatment, prevention or delay of progression of a HIV infection in a patient.

Compounds of the invention may exist in different forms, such as free acids, free bases, esters and other prodrugs, salts and tautomers, for example, and the disclosure includes all variant forms of these compounds.

The extent of protection includes counterfeit or fraudulent products which contain or purport to contain a compound of the invention irrespective of whether they do in fact contain such a compound and irrespective of whether any such compound is contained in a therapeutically effective amount.

Included in the scope of protection are packages which include a description or instructions which indicate that the package contains a species or pharmaceutical formulation of the invention and a product which is or comprises, or purports to be or comprise, such a formulation or species. Such packages may be, but are not necessarily, counterfeit or fraudulent.

Features, integers, characteristics, compounds, chemical moieties or groups described in conjunction with a particular aspect, embodiment or example of the invention are to be understood to be applicable to any other aspect, embodiment or example described herein unless incompatible therewith.

### DETAILED DESCRIPTION OF THE INVENTION

### Plant Species And Anti-HIV Activity

Justicia gendarussa Burm.f. belongs to Acanthaceae family. It is a bush up to about 0.3 m in height, and is found in Vietnam, China, Cambodia, India, Indonesia, Laos, Malaysia, Myanmar, Papua New Guinea, Philippines, Sri Lanka, Thailand (Flora of China). The fresh leaves have been traditionally used in these countries for treatment of muscle pains, broken/fractured bone, muscle sprains, cuts, hemiplegia, rheumatism, arthritis, headache and earache. The methanol extract of the whole plant of this plant was determined by us to be highly potent against HIV replication. The methanol extract made from the stem barks of this plant demonstrated capability to inhibit 50% HIV replication at a concentration of 0.04 g/mL. Further phytochemical study of this plant was thus conducted to identify the anti-HIV active compounds contained in this plant.

### Bioassay Directed-Fractionation

Bioassay directed-fractionation of the stem barks of J. gendarussa led to the isolation of the new aryl naphthalide lignan compounds of the present invention. “Bioassay-directed fractionation” is the sequential reduction of complex mixtures eventually to individual components. The extracts are tested for biological effects and subjected to one or several fractionation procedures. After each separation step the fractions are evaluated for biological activity for selection of active fractions for further investigation. When the complexity of the mixture is reduced to a few individual compounds, the fractions will be purified to obtain bioactive compounds, which are subjected to chemical identification and further bioactivity evaluation.

### Glycoside/Glycosidic Compound

The terms “glycoside” or “glycosidic compound” as used herein are interchangeable and includes reference to any of the class of compounds that yield a sugar and an aglycone upon hydrolysis.

### Aryl Naphthalide Lignan

The term “aryl naphthalide lignan” as used herein includes reference to a compound comprising the basic structure of 2,3-dimethyl-1-phenyl-naphthalene shown as below:

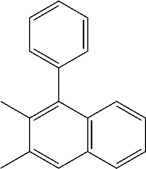 [SMILES: Cc1cc2ccccc2c(-c2ccccc2)c1C]

In one class of aryl naphthalide lignan compounds, the two methyl groups are forming a dihydro-furan-2-one ring, while in another class the two methyl groups are forming a dihydro-furan-3-one ring. Both classes of compounds have been found to be natural products.

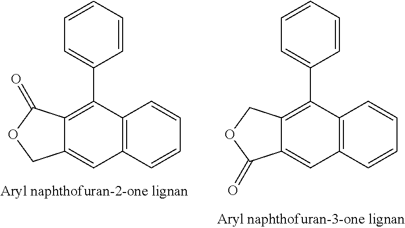 [SMILES: O=C1OCc2cc3ccccc3c(-c3ccccc3)c21 ; O=C1OCc2c1cc1ccccc1c2-c1ccccc1]

### Hydrocarbyl

The term “hydrocarbyl” as used herein includes reference to a moiety consisting exclusively of hydrogen and carbon atoms; such a moiety may comprise an aliphatic and/or an aromatic moiety. The moiety may comprise 1, 2, 3, 4, 5, 6, 7, 8, 9, 10, 11, 12, 13, 14, 15, 16, 17, 18, 19 or 20 carbon atoms. Examples of hydrocarbyl groups include C₁₋₆ alkyl (e.g. C₁, C₂, C₃ or C₄ alkyl, for example methyl, ethyl, propyl, isopropyl, n-butyl, sec-butyl or tert-butyl); C₁₋₆ alkyl substituted by aryl (e.g. benzyl) or by cycloalkyl (e.g. cyclopropylmethyl); cycloalkyl (e.g. cyclopropyl, cyclobutyl, cyclopentyl or cyclohexyl); aryl (e.g. phenyl, naphthyl or fluorenyl) and the like.

### Alkyl

The terms “alkyl” and “C₁₋₆ alkyl” as used herein include reference to a straight or branched chain alkyl moiety having 1, 2, 3, 4, 5 or 6 carbon atoms. This term includes reference to groups such as methyl, ethyl, propyl (n-propyl or isopropyl), butyl (n-butyl, sec-butyl or tert-butyl), pentyl, hexyl and the like. In particular, the alkyl moiety may have 1, 2, 3 or 4 carbon atoms.

### Alkenyl

The terms “alkenyl” and “C2-6 alkenyl” as used herein include reference to a straight or branched chain alkyl moiety having 2, 3, 4, 5 or 6 carbon atoms and having, in addition, at least one double bond, of either E or Z stereochemistry where applicable. This term includes reference to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

### Alkynyl

The terms “alkynyl” and “C₂-6 alkynyl” as used herein include reference to a straight or branched chain alkyl moiety having 2, 3, 4, 5 or 6 carbon atoms and having, in addition, at least one triple bond. This term includes reference to groups such as ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl and 3-hexynyl and the like.

### Alkoxy

The terms “alkoxy” and “C₁₋₆ alkoxy” as used herein include reference to —O-alkyl, wherein alkyl is straight or branched chain and comprises 1, 2, 3, 4, 5 or 6 carbon atoms. In one class of embodiments, alkoxy has 1, 2, 3 or 4 carbon atoms. This term includes reference to groups such as methoxy, ethoxy, propoxy, isopropoxy, butoxy, tert-butoxy, pentoxy, hexoxy and the like.

### Cycloalkyl

The term “cycloalkyl” as used herein includes reference to an alicyclic moiety having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbomyl, bicyclo[2.2.2]octyl and the like.

### Aryl

The term “aryl” as used herein includes reference to an aromatic ring system comprising 6, 7, 8, 9, 10, 11, 12, 13, 14, 15 or 16 ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

### Cyclic Group

“Cyclic group” means a ring or ring system, which may be unsaturated or partially unsaturated but is usually saturated, typically containing 5 to 13 ring-forming atoms, for example a 5- or 6-membered ring. It includes carbocyclyl and heterocyclyl moeities.

### Carbocyclyl

The term “carbocyclyl” as used herein includes reference to a saturated (e.g. cycloalkyl) or unsaturated (e.g. aryl) ring moiety having 3, 4, 5, 6, 7, 8, 9, 10, 11, 12, 13, 14, 15 or 16 carbon ring atoms. In particular, carbocyclyl includes a 3- to 10-membered ring or ring system and, in particular, 5- or 6-membered rings, which may be saturated or unsaturated. A carbocyclic moiety is, for example, selected from cyclopropyl, cyclobutyl, cyclopentyl, cyclohexyl, norbomyl, bicyclo[2.2.2]octyl, phenyl, naphthyl, fluorenyl, azulenyl, indenyl, anthryl and the like.

### Heterocyclyl

The term “heterocyclyl” as used herein includes reference to a saturated (e.g. heterocycloalkyl) or unsaturated (e.g. heteroaryl) heterocyclic ring moiety having from 3, 4, 5, 6, 7, 8, 9, 10, 11, 12, 13, 14, 15 or 16 ring atoms, at least one of which is selected from nitrogen, oxygen, phosphorus, silicon and sulphur. In particular, heterocyclyl includes a 3- to 10-membered ring or ring system and more particularly a 5- or 6-membered ring, which may be saturated or unsaturated.

A heterocyclic moiety is, for example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, pyrrolizidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4N-quinolizinyl, isoquinolyl, quinolyl, tetrahydroqu inolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazoiyl, β-carbolinyl, phenanthridinyl, acridinyl, perimidinyl, phenanthrolinyl, furazanyl, phenazinyl, phenothiazinyl, phenoxazinyl, chromenyl, isochromanyl, chromanyl and the like.

### Heterocycloalkyl

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulphur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like

### Heteroaryl

The term “heteroaryl” as used herein includes reference to an aromatic heterocyclic ring system having 5, 6, 7, 8, 9, 10, 11, 12, 13, 14, 15 or 16 ring atoms, at least one of which is selected from nitrogen, oxygen and sulphur. The group may be a polycyclic ring system, having two or more rings, at least one of which is aromatic, but is more often monocyclic. This term includes reference to groups such as pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl, pteridinyl and the like.

### Halogen

The term “halogen” as used herein includes reference to F, Cl, Br or I.

### Halogen Containing Moiety

The expression “halogen containing moiety” as used herein includes reference to a moiety comprising 1 to 30 plural valence atoms selected from carbon, nitrogen, oxygen and sulphur which moiety includes at least one halogen. The moiety may be hydrocarbyl for example C₁₋₆ alkyl or C₁₋₆ alkoxy, or carbocyclyl for example aryl.

### Substituted

The term “substituted” as used herein in reference to a moiety means that one or more, especially up to 5, more especially 1, 2 or 3, of the hydrogen atoms in said moiety are replaced independently of each other by the corresponding number of the described substituents. The term “optionally substituted” as used herein means substituted or un-substituted. It will, of course, be understood that substituents are only at positions where they are chemically possible, the person skilled in the art being able to decide (either experimentally or theoretically) without inappropriate effort whether a particular substitution is possible.

### Independently

Where two or more moieties are described as being “each independently” selected from a list of atoms or groups, this means that the moieties may be the same or different. The identity of each moiety is therefore independent of the identities of the one or more other moieties.

Embodiments of the invention are described below. Preferred features of each aspect of the invention are as for each of the other aspects mutatis mutandis. Moreover, it will be appreciated that the features specified in each embodiment may be combined with other specified features, to provide further embodiments.

### Compounds

The invention involves the use of aryl naphthalide lignan compounds, in particular glycosidic aryl naphthalide lignan compounds including derivatives of patentiflorin A, justiprocumin A or justiprocumin B. Preferably the aryl naphthalide lignan compounds are glycosidic aryl naphthalide lignan compounds.

In one embodiment, the invention provides compounds of the formula (I) or (II):

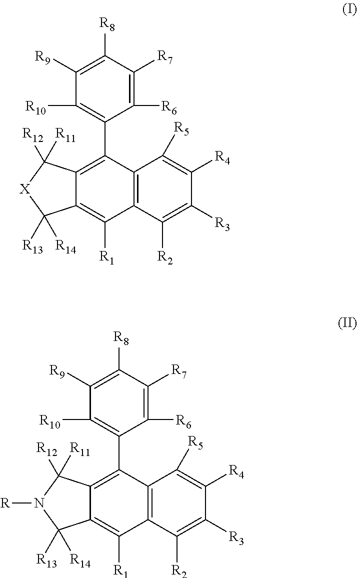

wherein
R₁, R₂, R₃, R₄, R₅, R₆, R₇, R₈, R₉ and R₁₀ are each independently hydrogen, halogen or a moiety comprising 1 to 30 plural valence atoms selected from carbon, nitrogen, oxygen and sulphur; or R₂ and R₃, R₃ and R₄, R₄ and R₅, R₆ and R₇, R₇ and R₈, R₈ and R₉ or R₉ and R₁₀ may be taken together with the carbon atoms to which they are attached to form a cyclic group which is optionally substituted with halogen or a moiety comprising 1 to 30 plural valence atoms selected from carbon, nitrogen, oxygen and sulphur; or at least one of R₁, R₂, R₃, R₄, R₅, R₆, R₇, R₈, R₉ and R₁₀ or a said cyclic group comprises a glycosidic group;
R₁₁ and R₁₂ may be taken together to form oxo; or while one of R₁₁ and R₁₂ is hydrogen or halogen, the other one of R₁₁ and R₁₂ is selected from R₁₅, —OR₁₅, —C(O)R₁₅ and —C(O)OR₁₆;
R₁₃ and R₁₄ may be taken together to form oxo; or while one of R₁₃ and R₁₄ is hydrogen or halogen, the other one of R₁₃ and R₁₄ is selected from R₁₅, —OR₁₅, —C(O)R₁₅ and —C(O)OR₁₅;
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3).

X is oxygen or sulphur;

R is hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3);
or a pharmaceutically acceptable salt or prodrug thereof.

The compound may comprise none, one or more (e.g. one or two) glycosidic groups, but usually comprises none or a single glycosidic group, and the glycosidic group typically at the 4- or 4′-position of the aryl naphthalide lignan. In a particular embodiment, R₁ comprises a glycosidic group. Of particular mention are compounds in which R₁ comprises a glycosidic group. Where other than a glycosidic group, R₁, R₂, R₃, R₄, R₅, R₆, R₇, R₈, R₉, and R₁₀ are often each independently selected from R₁₅, —OR₅, —C(O)R₁₅ and —C(O)OR₁₅, wherein:

R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈; and R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3).

In particular, one or more of R₁, R₂, R₃, R₄, R₅, R₆, R₇, R₈, R₉, and R₁₀ may be each independently selected from hydrogen, halogen, trifluoromethyl, R₁₅, —OR₁₅, —C(O)R₁₅ and —C(O)OR₁₅, wherein R₁₅ is hydrogen or selected from hydrocarbyl and —(CH₂)k-heterocyclyl, either of which is optionally substituted with 1, 2, 3, 4 or 5 substituents independently selected from halogen, cyano, amino, hydroxy, C₁₋₆ alkyl and C₁₋₆ alkoxy. In this regard, R₁₅ is especially hydrogen or C₁₋₆ alkyl optionally substituted with 1, 2 or 3 substituents independently selected from halogen, cyano, amino, hydroxy and C₁₋₆ alkoxy. Thus, one or more of R₁, R₂, R₃, R₄, R₅, R₆, R₇, R₈, R₉, and R₁₀ may be each independently selected from hydrogen, halogen, trifluoromethyl, hydroxy, C₁₋₆ alkyl and C₁₋₆ alkoxy, wherein C₁₄ alkyl and C₁₋₆ alkoxy are optionally substituted with 1, 2, 3, 4 or 5 substituents independently selected from, for example, halogen (e.g. fluorine or chlorine), cyano, amino, hydroxy and C₁₋₆ alkoxy.

As mentioned above, one or more of R2 and R3, R3 and R4, R4 and R5, R6 and R7, R7 and R₈, R₈ and R₉ or R₉ and R₁₀ may be taken together with the carbon atoms to which they are attached may form a cyclic group which is optionally substituted with halogen or a moiety comprising 1 to 30 plural valence atoms selected from carbon, nitrogen, oxygen and sulphur. The cyclic group may be a carbocyclyl (e.g. phenyl) or heterocyclyl (e.g. furanyl) group, either of which may be optionally substituted by, for example, one or more R₁₅, —OR₁₅, —C(O)R₅ and —C(O)OR₁₅. Alternatively, or additionally, a cyclic group so formed may comprise a glycosidic group, for example a sugar group.

The, or each, glycosidic group is generally a carbohydrate group, especially a monosaccharide, disaccharide, trisaccharide, tetrasaccharide or polysaccharide group, and may exist in various isomeric forms, for example α-D, α-L, β-D or β-L forms. The carbohydrate group may be optionally substituted with other type of substituents or even additional glycosidic groups. However, the total number of monosaccharide and substituted monosaccharide contained in the chemical structure of a compound may not exceed 30.

By way of illustration, a glycosidic group may be a group of the formula (i) or (ii):

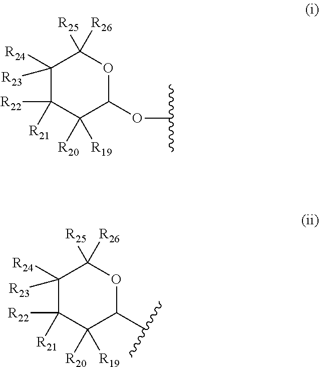

wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅, —C(O)OR₁₅, monosaccharide, substituted monosaccharide, disaccharide, substituted disaccharide, trisaccharide, substituted trisaccharide, tetrasaccharide and substituted tetrasaccharide;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅, —C(O)OR₁₅, monosaccharide, substituted monosaccharide, disaccharide, substituted disaccharide, trisaccharide, substituted trisaccharide, tetrasaccharide and substituted tetrasaccharide;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅, —C(O)OR₁₅, monosaccharide, substituted monosaccharide, disaccharide, substituted disaccharide, trisaccharide, substituted trisaccharide, tetrasaccharide and substituted tetrasaccharide;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅, —C(O)OR₁₅, —CH₂R₂₇ and —C(O)R₂₇;
R₂₇ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈, —N(R₁₇)S(O)₂R₁₈, monosaccharide, substituted monosaccharide, disaccharide, substituted disaccharide, trisaccharide, substituted trisaccharide, tetrasaccharide and substituted tetrasaccharide.

R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;

R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;

R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3);
A monosaccharide or a substituted monosaccharide is a group of the formula (iii) or (iv):

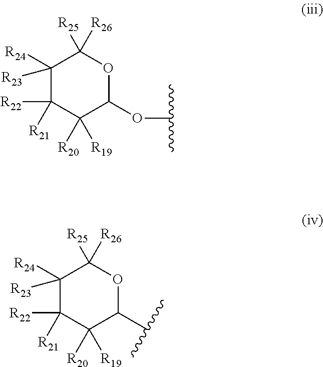

wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅ and —C(O)OR₁₅;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅ and —C(O)OR₁₅;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅ and —C(O)OR₁₅;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅ and —C(O)OR₁₅;
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3).

A disaccharide or a substituted disaccharide is a group of the formula (v) or (vi):

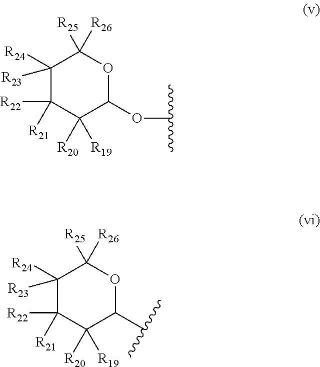

wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅, —C(O)OR₁₅, monosaccharide and substituted monosaccharide;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅, —C(O)OR₁₅, monosaccharide and substituted monosaccharide;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅, —C(O)OR₁₅, monosaccharide and substituted monosaccharide;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅, —C(O)OR₁₅, —CH₂R₂₇ and —C(O)R₂₇;
R₂₇ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈, —N(R₁₇)S(O)₂R₁₈, monosaccharide and substituted monosaccharide.
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3);

A trisaccharide or a substituted trisaccharide is a group of the formula (vii) or (viii):

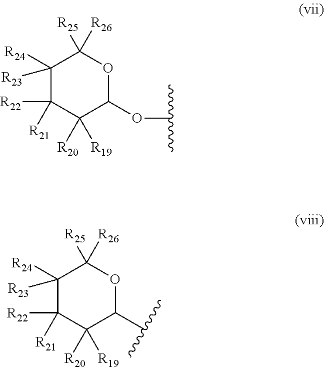

wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅, —C(O)OR₁₅, disaccharide and substituted disaccharide;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅, —C(O)OR₁₅, disaccharide and substituted disaccharide;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅, —C(O)OR₁₅, disaccharide and substituted disaccharide;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅, —C(O)OR₁₅, —CH₂R₂₇ and —C(O)R₂₇;
R₂₇ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈, —N(R₁₇)S(O)₂R₁₈, disaccharide and substituted disaccharide.
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3);

A tetrasaccharide or a substituted tetrasaccharide is a group of the formula (ix) or (x):

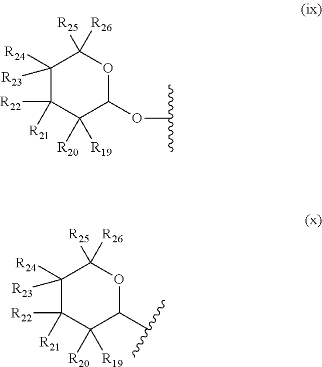

wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅, —C(O)OR₁₅, trisaccharide and substituted trisaccharide;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅, —C(O)OR₁₅, trisaccharide and substituted trisaccharide;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅, —C(O)OR₁₅, trisaccharide and substituted trisaccharide;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅, —C(O)OR₁₅, —CH₂R₂₇ and —C(O)R₂₇;
R₂₇ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈, —N(R₁₇)S(O)₂R₁₈, trisaccharide and substituted trisaccharide.
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6 (e.g. 1, 2 or 3);

Exemplary glycosidic groups include glucopyranoside, galactopyranoside, mannopyranoside, fucopyranoside, arabinopyranoside, glucopyranoside, galactopyranoside, glucuronide, lactopyranoside, xylopyranoside, glucosaminide, galactosaminide, alloside, lyxoside, taloside, threoside, riboside, fructoside, rhamnoside and guloside groups. More particularly, the glycosidic group may be selected from α-D-glucopyranoside, α-D-galactopyranoside, α-D-mannopyranoside, α-L-fucopyranoside, α-L-arabinopyranoside, β-D-glucopyranoside, β-D-galactopyranoside, β-D-glucuronide, β-D-lactopyranoside, β-D-xylopyranoside, β-D-glucosaminide, β-D-galactosaminide, β-D-alloside, β-D-lyxoside, β-D-taloside, β-D-threoside, β-D-riboside, β-D-fructoside, β-D-rhamnoside and β-L-guloside groups.

In particular compounds, R₁ is hydrogen, hydroxy, methoxy or glycosidic group; R₂, R₅, R₆, R₉ and R₁₀ are each independently hydrogen, hydroxy or methoxy; and R₃ and R₄ are each independently hydrogen, hydroxy, methoxy, or taken together with the carbon atoms to which they are attached form a [1,3]dioxole cyclic group; R₇ and R₈ are each independently hydrogen, hydroxy, methoxy, or taken together with the carbon atoms to which they are attached form a [1,3]dioxole cyclic group; R₁₁ and R₁₂ together form oxo; R₁₃ and R₁₄ are each independently hydrogen; X is oxygen.

Examples of compounds of the invention include those shown below. It will of course be appreciated that, where appropriate, each compound may be in the form of the free compound, an acid or base addition salt, or a prodrug.

### Examples

The root plus stem sample of Justicia gendarussa Burm.f. (Acanthaceae) was collected from Cuc Phuong National Park (Nho Quan District, Ninh Binh Province, Vietnam). Its methanol extract exhibited potent inhibition activity against HIV replication with an ICso50 value of 0.04 μ/mL. A 4.0 kg sample of the dried root plus stem of this plant was thus re-collected from the Cuc Phuong National Park in Vietnam in order to identify anti-HIV compounds. As a result, two new (1 and 2) and one known (3) aryl naphthalide lignans were isolated from this plant by bioassay-guided fractionation studies. In the structures of 1 and 2, Ac represents as an aceyl (—COCH₃) group, and Glu represents as a β-D-glucopyranosyl group.

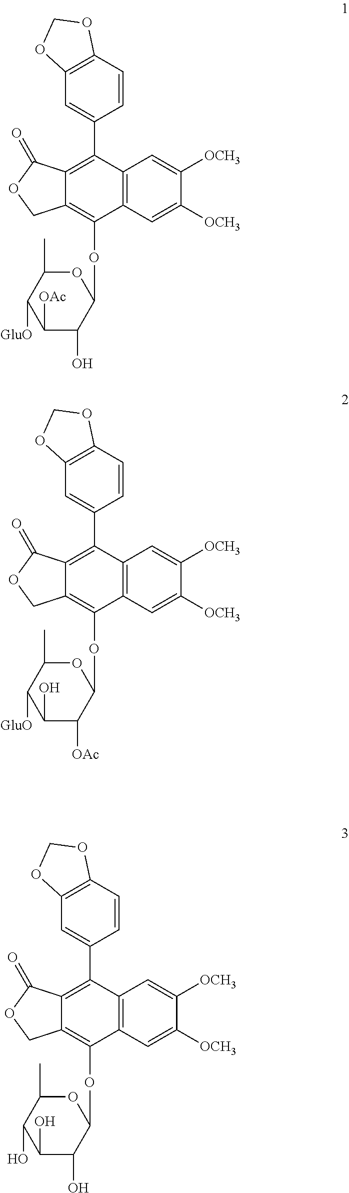

The recollected root plus stem sample (4.0 kg) of J. gendarussa was extracted with MeOH to afford an extract (155 g). Bioassy-directed fractionation of the MeOH extract by column chromatography on Si gel and RP-18 Si gel and subsequent preparative HPLC separation led to the isolation of two new aryl naphthalide lignans, justiprocumin A (1) and justiprocumin B (2), together with a known aryl naphthalide lignan, patentiflorin A (3).

Justiprocumin A (1) was obtained as a white powder with a molecular formula of C₃₅H₃₈O₁₇ by positive HRESIMS ([M+H]⁺ m/z 731.2217, calcd. 731.2187) and NMR studies (Tables 1 and 2). The compound was rapidly elucidated to be an aryl naphthalide lignan by comparison of its ¹H and ¹³C NMR data to those of aryl naphthalide lignan compounds. In combination of analysis 2D NMR data including ¹H-¹H COSY, HMQC and HMBC spectral data, 1 was determined as 9-(1,3-benzodioxol-5-yl)-4-[(3-O-acetyl-6-deoxy-4-O-β-D-glucopyranosyl-β-D-glucopyranosyl)oxy]-6,7-dimethoxy-naphtho[2,3-c]furan-1(3H)-one, and was given the trivial name justiprocumin A.

Justiprocumin B (2) was obtained as a white powder with the same molecular formula (C₃₅H₃₈O₁₇) to that of 1, which was determined by positive HRESIMS ([M+H]⁺ m/z 731.2216, calcd. 731.2187) and NMR studies (Tables 1 and 2). The structure of this compound is very similar to that of 1. Compound 2 differs from 1 only by the position of an acetyl group. In combination with analysis of 2D NMR data including ¹H-¹H COSY, HMQC and HMBC spectral data, 2 was determined as 9-(1,3-benzodioxol-5-yl)-4-[(2-O-acetyl-6-deoxy-4-O-β-D-glucopyranosyl-β-D-glucopyranosyl)oxy]-6,7-dimethoxy-naphtho[2,3-c]furan-1(3H)-one, and was given the trivial name justiprocumin B.

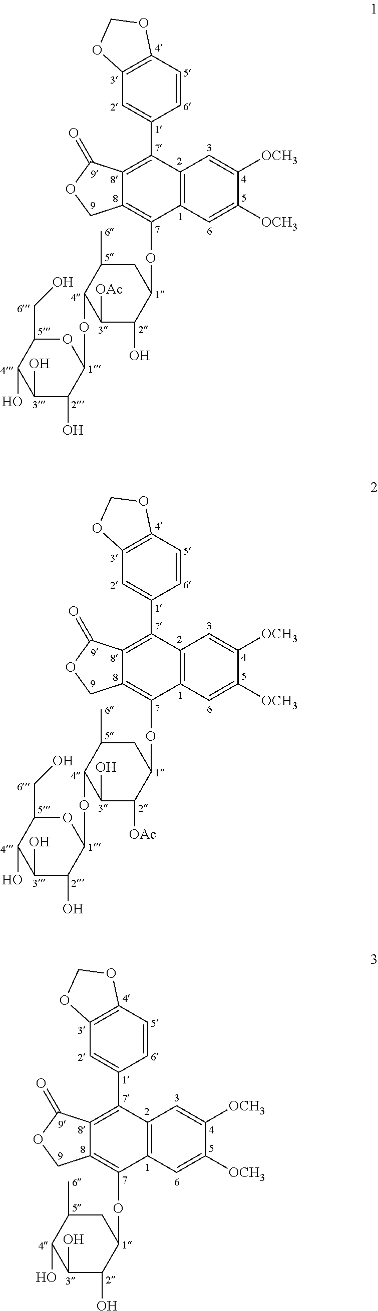

Compound 3 was obtained as the known compound patentiflorin A (Susplugas S, Nguyen V H, Bignon J, Thoison O, Kruczynski A, Sevenet T, Gueritte F. Journal of Natural Products 2005, 68: 734-738.) by analysis of its spectroscopic data (Tables 1 and 2), and a comparison of the data with those reported in literature. We further totally synthesized patentiflorin A. The synthesized patentiflorin A showed the same ¹H and ¹³C NMR spectral data as those of the natural patentiflorin A, which confirmed the chemical structure of patentiflorin A (Scheme 1).

TABLE 1
¹H NMR Spectral Data of Compounds 1-3 (1 and
2: 500 MHz; 3: 360 MHz; CD₃OD; J in Hz)

| | 1 | 2 | 3 |
|---|---|---|---|
| Position | δ (ppm) | | |
| H-3^{a} | 7.011 s | 6.962 s | 6.971 s |
| | 6.992 s | 6.954 s | 6.951 s |
| H-6^{a} | 0 | 7.496 s | 8.052 s |
| | 8.043 s | 7.493 s | 8.042 s |
| H-9a^{a} | 5.550 d (15.2) | 5.471 brs | 5.537 d (15.2) |
| | 5.546 d (15.2) | | 5.531 d (15.2) |
| H-9b^{a} | 5.418 d (15.1) | 5.471 brs | 5.410 d (15.2) |
| | 5.415 d (15.2) | | 5.406 d (15.4) |
| H-2′^{a} | 6.766 d (1.8) | 6.712 d (1.6) | 6.730 d (1.6) |
| | 6.725 overlap | 6.657 d (1.6) | 6.655 d (1.7) |
| H-5′^{a} | 6.929 d (7.8) | 6.908 d (7.9) | 6.897 d (7.9) |
| | 6.919 d (7.7) | | |
| H-6′^{a} | 6.723 dd (7.9, 1.9) | 6.685 dd (7.9, 1.7) | 6.682 dd (7.7, 1.8) |
| | 6.715 dd (7.7, 1.8) | 6.665 dd (7.8, 1.9) | 6.659 dd (7.8, 1.7) |
| H-1″^{a} | 4.852 d (7.9)^{f} | 5.184 d (8.1) | 4.761 d (7.8) |
| | 4.842 d (7.9)^{f} | 5.181 d (8.0) | 4.760 d (7.9) |
| H-2″^{a} | 3.862 dd (9.2, 7.7) | 5.266 dd (9.7, 7.9) | 3.638 dd (9.5, 8.1) |
| | 3.858 dd (9.4, 8.0) | 5.248 dd (9.8, 7.9) | 3.634 dd (9.1, 8.0) |
| H-3″ | 5.035 t (9.0) | 3.756 brt (9.0) | 3.408 t (9.1) |
| H-4″ | 3.603 t (9.4) | 3.453 t (9.1) | 3.316 t (9.5) |
| H-5″ | 3.522 dq (9.3, 5.9) | 3.668 m | 3.266 m |
| CH₃-6″^{a} | 1.425 d (6.0) | 1.465 d (6.2) | 1.303 d (6.0) |
| | 1.422 d (6.1) | 1.464 d (6.1) | 1.299 d (6.1) |
| H-1″′ | 4.319 d (7.7) | 4.431 d (7.8) | |
| H-2″′ | 3.100 dd (9.2, 8.0) | 3.229 dd (9.3, 8.0) | |
| H-3″′ | 3.325 t (8.8) | 3.377 t (9.0) | |
| H-4″′ | 3.220 dd (9.5, 8.7) | 3.272 brt (9.4) | |
| H-5″′ | 3.285 ddd (8.6, 6.2, 2.4) | 3.371 m | |
| H-6″′a | 3.918 dd (11.6, 2.1) | 3.914 dd (11.9, 2.1) | |
| H-6″′b | 3.667 dd (11.6, 6.2) | 3.645 dd (11.7, 6.1) | |
| 3′-OCH₂O- | 6.045 m | 6.036 m | 6.031 m |
| 4′^{a} | 6.029 m | 6.018 m | 6.019 m |
| 4-OCH₃^{a} | 3.683 s | 3.679 s | 3.670 s |
| | 3.675 s | 3.676 s | 3.664 s |
| 5-OCH₃^{a} | 3.961 s | 3.975 s | 3.974 s |
| | 3.959 s | 3.972 s | 3.970 s |
| 2″- | | 2.113 s | |
| OCOCH₃^{a} | | 2.111 s | |
| 3″-OCOCH₃ | 2.185 s | | |

^{a}The ¹H NMR signals were shown to be doubling due to some degree of hindered rotation of the aryl-naphthalene bond.

TABLE 2
¹³C NMR and DEPT Spectral Data of Compounds
1-3 (1 and 2: 500 MHz; 3: 360 MHz; CD₃OD)

| | | |
|---|---|---|
| 1 | 2 | 3 |

| | |
|---|---|
| Position | δ (ppm) |

| | | | |
|---|---|---|---|
| C-1 | 128.87 s | 127.36 s | 128.84 s |
| C-2 | 131.88 s | 131.60 s | 131.79 s |
| C-3 | 106.93 d | 106.91 d | 106.81 d |
| C-4 | 151.71 s | 151.77 s | 151.62 s |
| C-5 | 153.37 s | 153.25 s | 153.23 s |
| C-6 | 102.62 d | 101.86 d | 102.66 d |
| C-7 | 146.24 s | 145.96 s | 146.34 s |
| C-8^{a} | 132.18 s | 127.57 s | 132.22 s |
| | | 127.50 s | |
| C-9 | 69.05 t | 68.63 t | 69.10 t |
| C-1′ | 129.89 s | 129.85 s | 129.89 s |
| C-2′ | 111.75 d | 111.75 d | 111.75 d |
| C-3′ | 148.99 s | 148.93 s | 148.94 s |
| C-4′ | 148.99 s | 148.9 s | 148.94 s |
| C-5′ | 108.97 d | 108.94 d | |
| C-6′^{a} | 124.73 d | 124.74 d | 124.75 d |
| | | | 124.71 d |
| C-7′ | 137.74 s | 136.75 s | 137.50 s |
| C-8′^{a} | 119.96 s | 120.14 s | 119.89 s |
| | 119.92 s | | 119.84 s |
| C-9′ | 172.08 s | 171.76 s | 172.16 s |
| C-1″ | 106.35 d | 101.86 d | 106.68 d |
| C-2″ | 73.73 d | 75.18 d | 75.79 d |
| C-3″ | 76.79 d | 74.12 d | 77.80 d |
| C-4″ | 81.93 d | 85.77 d | 76.69 d |
| C-5″ | 73.03 d | 72.91 d | 73.63 d |
| C-6″ | 18.13 q | 18.07 q | 18.20 q |
| C-1 ″′ | 104.72 d | 105.01 d | |
| C-2″′ | 75.31 d | 75.07 d | |
| C-3″′ | 77.93 d | 77.93 d | |
| C-4″′ | 71.89 d | 71.51 d | |
| C-5″′ | 78.04 d | 78.22 d | |
| C-6″′ | 63.04 t | 62.59 t | |
| 3′-OCH₂O-4′ | 102.62 t | 102.9 t | 102.61 d |
| 4-OCH₃ | 55.99 q | 56.01 q | 55.97 q |
| 5-OCH₃ | 56.69 q | 56.68 q | 56.69 q |
| 2″-OCOCH₃ | | 21.30 q | |
| 3″-OCOCH₃ | 21.46 q | | |
| 2″-OCOCH₃ | | 171.76 s | |
| 3″-OCOCH₃ | 173.11 s | | |

^{a}The ¹³C NMR signals were shown to be doubling due to some degree of hindered rotation of the aryl-naphthalene bond.

Our “Anti-HIV Post-Entry” protocol has been used for initial evaluation of anti-HIV activity of fractions and isolated compounds. By using this protocol, all three isolated compounds (1-3) exhibited potent inhibition of HIV replication. Patentiflorin A (3) demonstrated anti-HIV activity with an IC₅₀ value of less than 10 ng/mL, and both justiprocumins A (1) and B (2) inhibited HIV replication by more than 100% at a concentration of 1 μg/mL.

Justiprocumins B (2) and patentiflorin A (3) were further evaluated against a broad spectrum of HIV strains including four HIV1 clinical strains, BAL and SF162 (M-tropic), BAL (T-tropic), and 89.6 (a dual tropic strain). In these experiments, AZT was used as a positive control. Justiprocumin A (1) was not tested since it is structurally similar to justiprocumin B (2) and exhibited a similar inhibition profile to that of Justiprocumin B (2) using the “One-Stone-Two Birds” protocol. It is clear that justiprocumin B (2) and patentification A (3), like AZT, could inhibit the particle production of all four HIV-1 strains effectively in a dose-dependent manner. Justiprocumin B (2) gave an IC₅₀ value of 14-21 nM, and patentification A (3) an IC₅₀ value of 24-37 nM, compared to 77-95 nM for AZT, depending on the strains (Table 3).

TABLE 3
Anti-HIV activity of justiprocumin B (2) and
patentiflorin A (3) against four HIV strains.

| | IC₅₀ (nM) | | | |
|---|---|---|---|---|
| | Bal: M- | 89.6: Dual- | SF162: M- | Lav.04: T- |
| | Tropic | Tropic | Tropic | Tropic |
| Justiprocumin | 15 | 15 | 14 | 21 |
| B (2) | | | | |
| Natural patentiflorin | 30 | 32 | 37 | 24 |
| A (3) | | | | |
| Synthetic patentiflorin | 32 | 31 | 30 | 32 |
| A (3) | | | | |
| AZT | 77 | 95 | 85 | 79 |

As shown above, these identified potent anti-HIV compounds are structurally similar. To determine their toxicity, patentiflorin A was chosen to be evaluated for its toxicity against the A549 and Hela cell lines. At a concentration of 10 μg/mL, patentiflorin A exhibited no apparent cytotoxicity against A549 and Hela cell lines.

To confirm the anti-HIV activity of the aryl naphthalide lignan glycosides, we synthesized, de novo, one (patentiflorin A) of the aryl naphthalide lignan glycosides. Patentiflorin A was isolated as a potent anti-HIV aryl naphthalide lignan glycoside compound from the root plus stem sample of Justicia gendarussa through bioassay-guided fractionation. In order to confirm its anti-HIV activity and obtain a sufficient amount of this compound for further biological study, we undertook to totally synthesize this compound, de novo. Scheme 1 showed our successful route in synthesizing patentiflorin A. The spectral data (MS and NMR) of the synthetic compound being the same as those of the natural isolate confirmed that the two compounds are of the same structure. We further determined that the synthetic patentiflorin A (SF85) showed similar anti-HIV activity as that of the isolated patentiflorin A. We further evaluated inhibition of natural patentiflorin A and synthetic patentiflorin A against the four HIV-1 strains (Table 3). Bioassay showed that the synthetic patentiflorin A not only retained the anti-HIV potency in comparison of the natural patentiflorin A, but also had a better therapeutic index in comparison of the natural compound (SI>2,500). We further investigated this compound against HIV gene expression of the R/U5 and U5/gag transcripts. Our data showed that the compound acts as an effective inhibitor of HIV reverse transcription. Thus, we have confirmed the anti-HIV activity of the aryl naphthalide lignan compounds through chemical synthesis and biological approach.

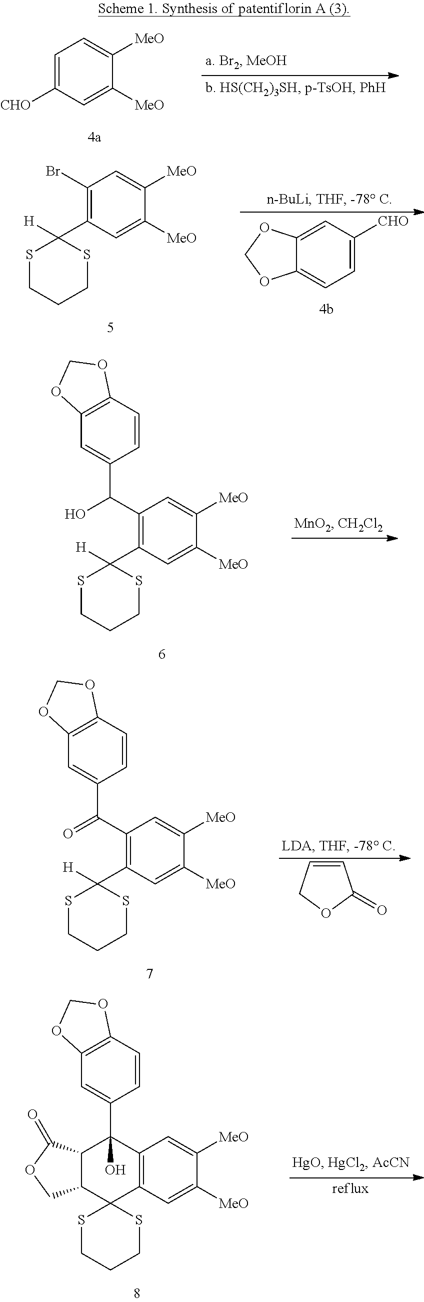

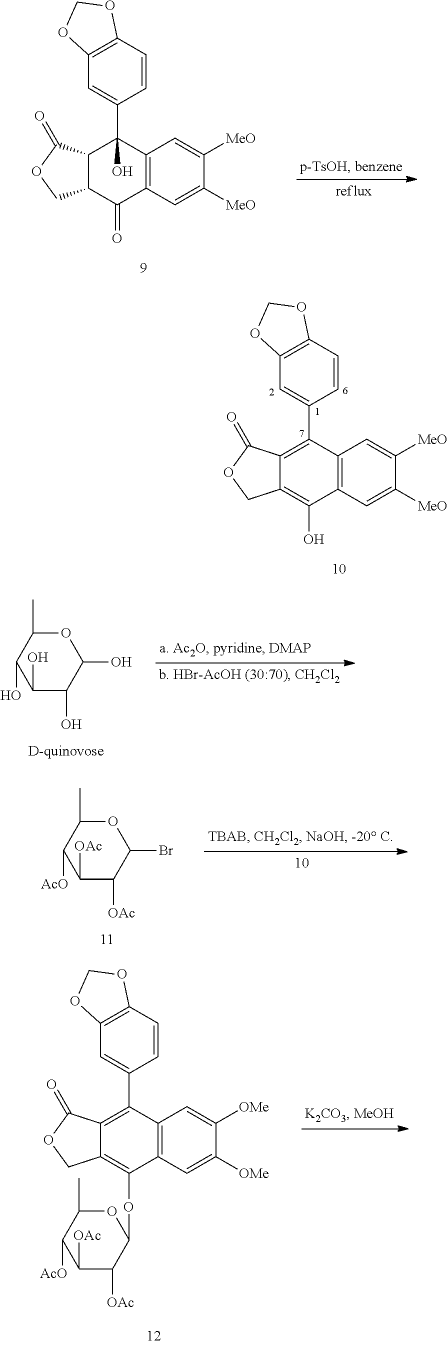

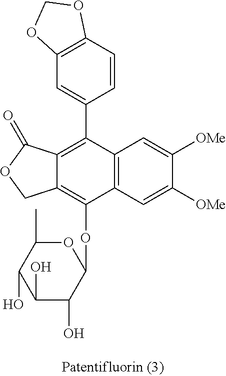

General Experimental Procedures.

Optical rotations were measured on a Perkin-Elmer model 241 polarimeter. IR spectra were run on a Jasco FT/IR-410 spectrometer, equipped with a Specac Silver Gate ATR system by applying a film on a Germanium plate. 1D and 2D NMR spectra were recorded on a Bruker Avance-360 or Avance-500 MHz spectrometer. Column chromatography was carried out on silica gel (200-400 mesh, Natland International Corporation), and reversed-phase flash chromatography was accomplished with RP-18 silica gel (40-63μ, EM Science). Reversed-phase HPLC was carried out on a Waters 600E Delivery System pump, equipped with a Waters 996 photodiode detector, and a Phenomenex LUNA C18 column (10μ, 250×50 mm), which also resulted in extracting UV spectral data of each purified compound. Thin-layer chromatography was performed on Whatman glass-backed plates coated with 0.25 mm layers of Silica gel 60. HRTOFMS spectra were recorded on a ThermoFinnigan LTQFT spectrometer.

Plant Material.

The initial collection of root and stem sample (SV5614) of Justicia procumbens L. (Acanthaceae) was made in Phu Lai at Hoa Binh province side of Cuc Phuong National Park, with geographic coordinate readings of 20°21′633″N 105° 38′257′E, and voucher herbarium specimen Mai Van Xinh 742. A larger amount of the plant sample for the current isolation work, consisting of the same plant part (bark and stem), sample SVA5614 (4.0 kg), was subsequently re-collected from plants located in the same area. The recollection of root and stem sample (SVA5614) of J. gendarussa was made near Cave of Prehistoric Man close to hotspot 33 of Cuc Phuong National Park, with geographic coordinate readings of 20°17′ 516″N, 105°40′211″E. Voucher specimens (N.M. Cuong #2162) have been deposited at the Herbarium of Cuc Phuong National Park.

Extraction and Isolation.

The dried, milled plant material (4.0 kg) was extracted with methanol to afford 155 g of extract. A small portion of the methanol extract (5.5 g) was rapidly fractionated to 22 fractions (Fa1-Fa22) over a silica gel (10 g) column eluting with gradient CHCl₃, CHCl₃/Me₂CO, and CHCl₃/MeOH solutions. Anti-HIV evaluation of the fractions determined that the fractions Fa14-Fa18 inhibited HIV replication by more than 95% at a concentration of 0.1 μg/mL without apparent toxicity at 20 μg/mL. HPLC separation of Fa16 led to the isolation of justiprocumins A (1) and B (2). Scale-up fractionation of SVA5614 was then carried out in attempt to isolate other anti-HIV active compounds in this plant. The methanol extract (140 g) was absorbed in 214 g silica gel, and chromatographed over a silica gel column (1545 g). The column was developed by gradient elution with CHCl₃, CHCl₃/Me₂CO, and CHCl₃/Me₂CO/MeOH solutions to afford 40 fractions. Fraction F26 showed very similar TLC and HPLC profiles to those of Fa16. A portion of F26 was thus subjected preparative HPLC separation to yield 8 fractions (F41-F48). Bioassay showed that the fraction F47 was able to inhibit HIV replication by more than 90% at a concentration of 0.2 μg/mL. HPLC separation of F47 led to the isolation of patentiflorin A (3).

Justiprocumins A (1).

White powder, [α]_{D}²⁰ −26.2° (c 0.55, MeOH); UV λmax [AU (absorbance units)]=201.6 (0.77), 225.2 (0.41), 261.5 (1.01), 294.0 (0.19), 314.9 (0.21), 356.1 (0.07) nm; IR (film) vₘₐₓ 3421 (br), 2924, 1745, 1622, 1507, 1480, 1456, 1435, 1391, 1341, 1262, 1229, 1168, 1066, 1034, 930, 865, 770 cm⁻¹; ¹H and ¹³C NMR data, see Tables 1 and 2; positive HRESIMS [M+H]⁺ m/z 731.2217 [M+H]⁺ (calcd. for C₃₅H₃₈O₁₇, 731.2187).

Justiprocumins B (2).

White powder, [α]_{D}²⁰-32.00 (c 0.74, MeOH); UV λmax [AU (absorbance units)]=201.6 (1.16), 226.5 (0.63), 261.5 (1.41), 293.6 (0.29), 314.9 (0.31), 356.0 (0.14) nm; IR (film) vₘₐₓ 3422 (br), 2924, 1748, 1651, 1621, 1558, 1541, 1507, 1476, 1456, 1435, 1450, 1262, 1227, 1168, 1068, 1035, 931, 863, 770 cm⁻¹; ¹H and ¹³C NMR data, see Tables 1 and 2; positive HRESIMS [M+H]⁺ m/z 731.2216 [M+H]⁺ (calcd. for C₃₅H₃₈O₁₇, 731.2187).

Patentiflorin A (3).

White powder, [α]_{D}²⁰ −20.4° (c 1.61, MeOH); UV λmax [AU (absorbance units)]=200.4 (1.06), 225.1 (0.54), 261.5 (1.29), 293.9 (0.24), 314.9 (0.26), 355.0 (0.10) nm; ¹H and ¹³C NMR data, see Tables 1 and 2.

Total Synthesis of Patentiflorin A (3) (Scheme 1).

To a solution of veratral (4a) (63.2 mmol) in methanol (MeOH, 250 mL), a solution of bromine (Br₂, 69.5 mmol) was added slowly. The reaction was allowed to stir at room temperature for 6 hr to afford 2-bromo-veratral (13.4 g, 86%). 2-Bromo-veratral (50.3 mmol) was then dissolved in 300 mL benzene, to which, 1,3-propanedithiol (50.4 mmol) and p-toluenesulfonic acid (p-TsOH, 2.5 mmol) were added. The reaction mixture was allowed to be stirred at reflux for 10 hr then ambient temperature for 48 hr to yield 5 (15.6 g, 93%). To a stirred solution of compound 5 (46.6 mmol) in tetrahydrofuran (THF, 150 mL) at −78° C. was added n-butyllithium (n-BuLi) (1.6 M solution in hexanes, 69.9 mmol) in 1 hr, followed by addition of a solution of piperonal (4b) (55.9 mmol) in THF (30 mL). The reaction was allowed for a further 2 hr and then 3 hr at room temperature to yield 6 (9.5 g, 50%). Compound 6 (20.7 mmol) in CH₂Cl₂ (200 mL) was added a suspension of activated manganese dioxide (MnO₂, 345 mmol) at room temperature. The reaction was allowed for 16 hr, and was then filtered through a plug of celite to afford the oxidative product 7 (8.2 g, 98%). n-Butyllithium (1.6M solution in hexanes, 100 mmol) was added dropwise over 5 min to a cooled (−78° C.) solution of diisopropylamine (100 mmol) in THF (43 mL) under Argon to make fresh lithium diisopropylamide (LDA) solution. The solution was warmed to ambient temperature over 30 min and 25.7 mL added, dropwise via syringe over 3 min to a cooled (−78° C.) THF solution (60 mL) of the dithiane 7 (14.29 mmol). After 40 min, 2,5-dihydrofuranone (17.1 mmol) as a solution in THF (10 mL) was added over 1 min. The reaction is warmed to ambient temperature for 1 hr to afford 8 (3.95 g, 57%). A solution of compound 8 (0.65 mmol), mercury oxide (HgO, 0.71 mmol) and mercury chloride (HgCl₂, 1.43 mmol) in 84% acetonitrile (AcCN) aqueous (25 mL) was heated to reflux for 3 hr to afford 9 (134 mg, 52%). The ketone 9 (0.34 mmol) and p-TsOH (0.19 mmol) were heated to reflux in benzene (150 mL) for 16 hr to yield the desired compound diphyllin (10, 106 mg, 83%). To the solution of D-quinovose (0.61 mmol) in pyridine (5 mL) was added 4-dimethylaminopyridine (DMAP, 0.06 mmol) and acetic anhydride (Ac₂O, 1.5 mL) at room temperature, and the mixture was stirred overnight and quenched with MeOH (1 mL) to afford 1,2,3,4-tetraacetyl-D-quinovose. Without further separation, the tetraacetate was dissolved in glacial acetic acid (AcOH, 1 mL), and 1.5 mL hydrobromic acid (HBr) (33% in AcOH) was added to the solution slowly at room temperature. The reaction was allowed for 15 min to afford 11 (215 mg, 97%). To a solution of TBAB (tetrabutylammonium bromide, 0.95 mmol) and diphyllin (10, 0.95 mmol) in 15 mL of dichloromethane (CH₂Cl₂) was added aqueous 0.1 M sodium hydroxide (NaOH, 20 mL). After stirring for 10 min at 40° C., compound 11 (0.61 mmol) was then added, and the two-phase reaction was stirred for 6 hr at 40° C. to afford the solid 12. Without further separation, the solid 12 (538 mg) was dissolved in MeOH (10 mL), and potassium carbonate (K₂CO₃, 1.0 mmol) was then added. The reaction was allowed for 1 hr to afford patentiflorin A (3) (122 mg).

Anti-HIV Evaluation Using Post-Entry Protocol.

This protocol allows us to identify potential inhibitors for HIV replication (post-entry steps). In this system, the HIV vector pNL4-3.Luc. R. E (Connor R I, Chen B K, Choe S, Landau N R. Virology 1995; 206: 935-44; and He J, Choe S, Walker R, Di Marzio P, Morgan P O, Landau N R. Journal of Virology 1995; 69: 6705-11.), obtained through the AIDS Research and Reference Reagent Program (Division of AIDS, NIAID, NIH), was co-transfected with the H5N1 HA and NA constructs to generate HIV virions with bird flu HA on the viral surface (HIV/HA). This pNL4-3 was derived from an infectious molecular clone of an SI, T-tropic virus (Michael N L, Nelson J A, KewalRamani V N, Chang G, O'Brien S J, Mascola J R, Volsky B, Louder M, White G C 2nd, Littman D R, Swanstrom R, O+Brien T R. Journal of Virology 1998; 72: 6040-7.), and is replication deficient since the HIV is Env⁻ and Vpr⁻. Also the luciferase gene (luc) carried by this recombinant HIV vector serves as the reporter for HIV replication (reverse transcription, integration and HIV gene expression). The evaluation principle is that the level of the luciferase activity in the cells should be proportional to the level of viral entry and replication. If a compound (or fraction) can interfere with HIV replication/or HA-mediated viral entry, the level of the luciferase activity in the infected cells will be reduced. Thus, using this protocol, we were able to identify fractions or compounds capable of inhibiting HIV replication. The fractions or compounds were evaluated as follows. The stock HIV/HA virions (approximately 2×10⁶ relative light units, or RLUs, on the target cells) were mixed with the individual extract first, and the mixture was incubated with the target cells in 24 well plates (human lung cell line A549 was used since it is susceptible to HA-mediated viral entry). The final concentration of the extract was 20 μg/ml. Forty-eight hours post-infection, the target cells were lysed and the luciferase activity was determined.

Anti-HIV Evaluation Using Four HIV1 Clinical Strains.

Four HIV1 clinical strains, BAL and SF162 (M-tropic), BAL (T-tropic), and 89.6 (a dual tropic strain), were used in the current study. Here a standardized human peripheral blood mononuclear cell culture (PBMC) assay was used to determine the compound susceptibility of these HIV-1 strains. AZT, an anti-HIV drug in clinical use, was used as a positive control. Briefly, human PBMCs were collected from a donor and stimulated for seven days. The preparations (compound or fraction) were then added to the cultured cells at a wide range of concentrations, and the different HIV-1 strains were used to challenge the cultured cells using 96-well plates. After seven-days of incubation, the supernatants were collected and the HIV p24 levels of the infected cells were determined using a p24 antigen ELISA. The ICsos were calculated by comparing p24 antigen values for the compounds (fraction)-containing wells with those for no drug control wells. In these experiments, AZT was used as positive controls.

Evaluation of Toxicity.

Approximately 5000 cells seeded in a 96-well tissue culture plate in DMEM supplemented with FBS and penicillin/streptomycin. DMSO alone or compounds in 100% DMSO were added on the following day at appropriate concentrations and incubated with cells at 37° C. in 5% CO₂ for 24 hr. After 24 hr, all media were removed and replaced with 100 μL fresh complete DMEM. After 48 hr post initial addition of DMSO or compound, 20 μL of CellTiter 96 Aqueous One Solution was added per well. After gentle mixing, plates were incubated at 37° C. in 5% CO₂ for 4 hr. 25 μL of a 10% SDS solution was added per well and plates were stored at room temperature for approximately 12 hr. Absorbance was then measured at 450 nm using a plate reader.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

When a group of materials, compositions, components or compounds is disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

All references cited herein are hereby incorporated by reference in their entirety to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional cells, and additional uses of the invention. All headings used herein are for convenience only. All patents and publications mentioned in the specification are indicative of the levels of skill of those skilled in the art to which the invention pertains, and are herein incorporated by reference to the same extent as if each individual publication, patent or patent application was specifically and individually indicated to be incorporated by reference. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

1. A method of treating, preventing or delaying the progression of a HIV infection in a patient, comprising administering a therapeutically effective amount of a compound having the formula (I) or (II): 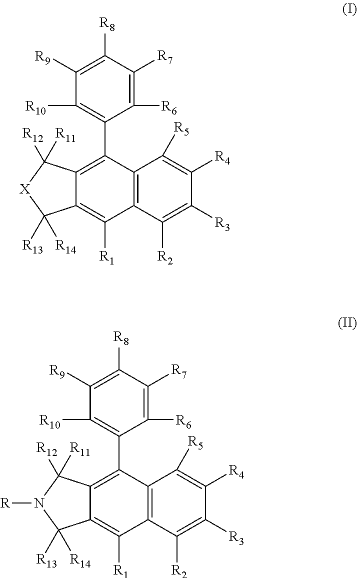
wherein
R₁, R₂, R₃, R₄, R₅, R₆, R₇, R₈, R₉ and R₁₀ are each independently hydrogen, halogen or a moiety comprising 1 to 30 plural valence atoms selected from carbon, nitrogen, oxygen and sulphur, or R₂ and R₃, R₃ and R₄, R₄ and R₅, R₆ and R₇, R₇ and R₈, R₈ and R₉ or R₉ and R₁₀ may be taken together with the carbon atoms to which they are attached to form a cyclic group which is optionally substituted with halogen or a moiety comprising 1 to 30 plural valence atoms selected from carbon, nitrogen, oxygen and sulphur, or at least one of R₁, R₂, R₃, R₅, R₆, R₇, R₈, R₉ and R₁₀ or a said cyclic group comprises a glycosidic group;
R₁₁ and R₁₂ may be taken together to form oxo; or while one of R₁₁ and R₁₂ is hydrogen or halogen, the other one of R₁₁ and R₁₂ is selected from R₁₅, —OR₁₅, —C(O)R₁₅ and —C(O)OR₁₅;
R₁₃ and R₁₄ may be taken together to form oxo; or while one of R₁₃ and R₁₄ is hydrogen or halogen, the other one of R₁₃ and R₁₄ is selected from R₁₅, —OR₁₅, —C(O)R₅ and —C(O)OR₁₅;
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6;
X is nitrogen, oxygen or sulphur;
R is hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and Ct alkoxy; wherein k is an integer between 1 and 6;
or a pharmaceutically acceptable salt or prodrug thereof;
while R₁, R₂, R₅, R₆, R₉, R₁₀, R₁₁ and R₁₂ are each independently hydrogen, X is oxygen, and R₁₃ and R₁₄ taken together to form oxo, R₃, R₄, R₇ and R₈ each should not be OCH₃;
while R₁, R₂, R₅, R₆, R₉, R₁₀, R₁₁ and R₁₂ are each independently hydrogen, X is oxygen, and R₁₃ and R₁₄ taken together to form oxo, R₃, R₄, R₇ and R₈ each should not be OH;
while R₁, R₂, R₅, R₈, R₉, R₁₀, R₁₁ and R₁₂ are each independently hydrogen, X is oxygen, and R₁₃ and R₁₄ taken together to form oxo, R₃ and R₄ or R₇ and R₈ taken together with the carbon atoms to which they are attached should not form a [1,3]dioxolo group;
while R₁, R₂, R₅, R₆, R₉, R₁₀, R₁₁, R₁₂, R₁₃ and R₁₄ are each independently hydrogen, and R₃, R₄, R₇ and R₈ are each independently OCH₃, R should not be CH₃, CH₂CH₂CH₃, CH(CH₃)CH₃ or CH₂CH₂CH₂CH₃.

2. The method according to claim 1, wherein the compound comprises none, one or more glycosidic groups.

3. The method according to claim 1, wherein the compound comprises a glycosidic group which is a monosaccharide, substituted monosaccharide, disaccharide, substituted disaccharide, trisaccharide, substituted trisaccharide, tetrasaccharide or substituted tetrasaccharide group.

4. The method according to claim 3, wherein the glycosidic group is selected from a group of the formula (i) or (ii): 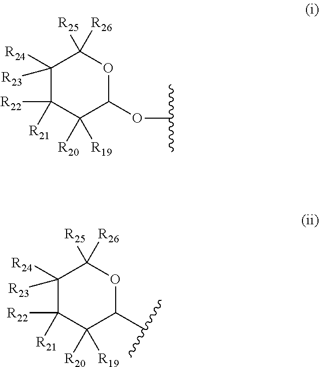
wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅, —C(O)OR₁₅, monosaccharide, substituted monosaccharide, disaccharide, substituted disaccharide, trisaccharide, substituted trisaccharide, tetrasaccharide and substituted tetrasaccharide;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅, —C(O)OR₁₅, monosaccharide, substituted monosaccharide, disaccharide, substituted disaccharide, trisaccharide, substituted trisaccharide, tetrasaccharide and substituted tetrasaccharide;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅, —C(O)OR₁₅, monosaccharide, substituted monosaccharide, disaccharide, substituted disaccharide, trisaccharide, substituted trisaccharide, tetrasaccharide and substituted tetrasaccharide;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅, —C(O)OR₁₅, —CH₂R₂₇ and —C(O)R₂₇;
R₂₇ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈, —N(R₁₇)S(O)₂R₁₈, monosaccharide, substituted monosaccharide, disaccharide, substituted disaccharide, trisaccharide, substituted trisaccharide, tetrasaccharide and substituted tetrasaccharide;
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6.

5. The method according to claim 3, wherein the monosaccharide or the substituted monosaccharide group is selected from a group of the formula (iii) or (iv): 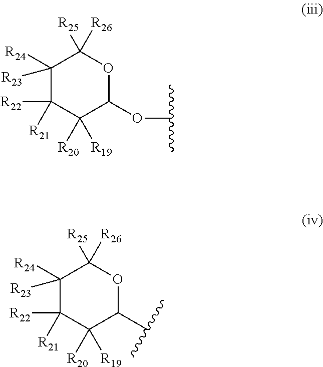
wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅ and —C(O)OR₁₅;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅ and —C(O)OR₁₅;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅ and —C(O)OR₁₅;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅ and —C(O)OR₁₅;
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6.

6. The method according to claim 3, wherein the disaccharide or the substituted disaccharide group is selected from a group of the formula (v) or (vi): 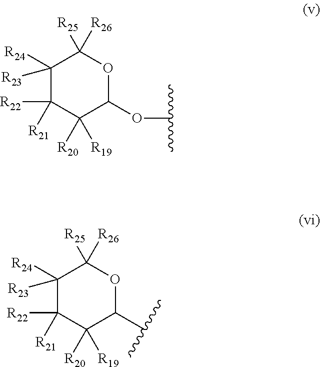
wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅, —C(O)OR₁₅, monosaccharide and substituted monosaccharide;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅, —C(O)OR₁₅, monosaccharide and substituted monosaccharide;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅, —C(O)OR₁₅, monosaccharide and substituted monosaccharide;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅, —C(O)OR₁₅, —CH₂R₂₇ and —C(O)R₂₇;
R₂₇ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₇)C(O)R₁₈, —N(R₁₇)S(O)₂R₁₈, monosaccharide and substituted monosaccharide;
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6.

7. The method according to claim 3, wherein the trisaccharide or the substituted trisaccharide group is selected from a group of the formula (vii) or (viii): 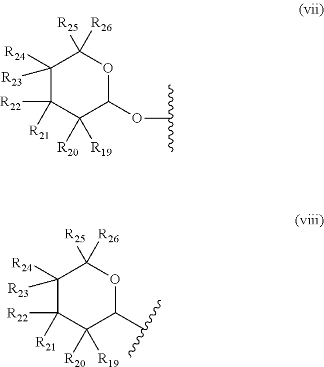
wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅, —C(O)OR₁₅, disaccharide and substituted disaccharide;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅, —C(O)OR₁₅, disaccharide and substituted disaccharide;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅, —C(O)OR₁₅, disaccharide and substituted disaccharide;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅, —C(O)OR₁₅, —CH₂R₂₇ and —C(O)R₂₇;
R₂₇ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈, —N(R₁₇)S(O)₂R₁₈, disaccharide and substituted disaccharide;
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocyclyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6.

8. The method according to claim 3, wherein the tetrasaccharide or the substituted tetrasaccharide group is selected from a group of the formula (ix) or (x): 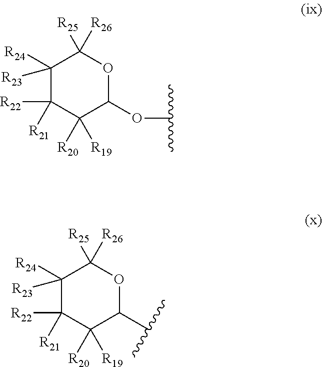
wherein
R₁₉ and R₂₀ may be taken together to form oxo; or while one of R₁₉ and R₂₀ is hydrogen or halogen, the other one of R₁₉ and R₂₀ is selected from R₁₅, —OR₁₅, —C(O)R₁₅, —C(O)OR₁₅, trisaccharide and substituted trisaccharide;
R₂₁ and R₂₂ may be taken together to form oxo; or while one of R₂₁ and R₂₂ is hydrogen or halogen, the other one of R₂₁ and R₂₂ is selected from R₁₅, —OR₁₅, —C(O)R₁₅, —C(O)OR₁₅, trisaccharide and substituted trisaccharide;
R₂₃ and R₂₄ may be taken together to form oxo; or while one of R₂₃ and R₂₄ is hydrogen or halogen, the other one of R₂₃ and R₂₄ is selected from R₁₅, —OR₁₅, —C(O)R₁₅, —C(O)OR₁₅, trisaccharide and substituted trisaccharide;
R₂₅ and R₂₆ may be taken together to form oxo; or while one of R₂₅ and R₂₆ is hydrogen or halogen, the other one of R₂₅ and R₂₆ is selected from R₁₅, —OR₁₅, —C(O)R₁₅, —C(O)OR₁₅, —CH₂R₂₇ and —C(O)R₂₇;
R₂₇ is independently selected from hydrogen, halogen, trifluoromethyl, cyano, nitro, hydrocarbyl optionally substituted with 1, 2, 3, 4 or 5 R₁₆, —(CH₂)ₖ-heterocycyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈, —N(R₁₇)S(O)₂R₁₈, trisaccharide and substituted trisaccharide;
R₁₅ is independently selected from hydrogen, halogen, trifluoromethyl, cyano, nitro, hydrocarbyl optionally substituted with 1, 2, 3, 4 or 5 R₁₆, —(CH₂)ₖ-heterocycyl optionally substituted with 1, 2, 3, 4 or 5 R₁₆, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₆ is independently selected from halogen, trifluoromethyl, cyano, nitro, oxo, ═NR₁₇, —OR₁₇, —C(O)R₁₈, —C(O)N(R₁₇)R₁₈, —C(O)OR₁₇, —OC(O)R₁₇, —S(O)₂R₁₇, —S(O)₂N(R₁₇)R₁₈, —N(R₁₇)R₁₈, —N(R₁₇)N(R₁₇)R₁₈, —N(R₁₇)C(O)R₁₈ and —N(R₁₇)S(O)₂R₁₈;
R₁₇ and R₁₈ are each independently hydrogen or selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy; wherein k is an integer between 1 and 6.

9. The method according to claim 1, wherein the compound is selected from: 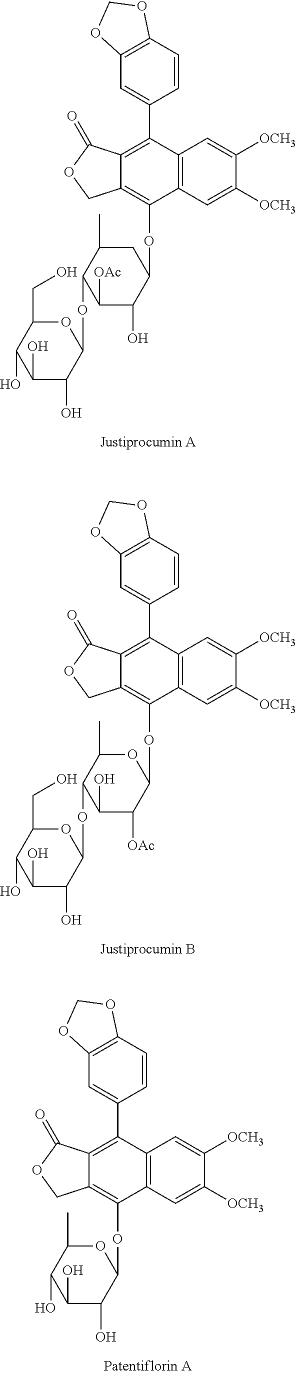
or a pharmaceutically acceptable salt or prodrug thereof.

10. (canceled)

11. (canceled)
